(19) United States
(12) Patent Application Publication
(10) Pub. No.: US 2010/0085160 A1
(43) Pub. Date: Apr. 8, 2010

(54) Systems and Methods for Zero-Power Security

(75) Inventor: Kevin Edward Fu, Hadley, MA (US)

Correspondence Address: MIRICK, O'CONNELL, DEMALLIE & LOUGEE, LLP, 1700 WEST PARK DRIVE, WESTBOROUGH, MA, 01581, US

(73) Assignee: University of Massachusetts, Boston, MA (US)

(21) Appl. No.: 12/541,751

(22) Filed: Aug. 14, 2009

Related U.S. Application Data
(60) Provisional application No. 61/102,677, filed on Oct. 3, 2008.

(51) Int. Cl. H04K 1/00; A61N 1/08; H04Q 5/22

(52) U.S. Cl. 340/10.1; 607/60; 380/270

(57) ABSTRACT

The present invention provides systems and methods for utilizing zero-power, energy-harvesting computational modules to provide secure and reprogrammable wireless communications with vulnerable devices comprising integrated circuits (ICs), including active implantable medical devices, electronic lock and key systems, credit cards, access cards, identification cards and passports. The zero-power, energy-harvesting computational modules are powered by radio signals received from an interrogator, and requests from the interrogator are authenticated using an encrypted challenge-response mechanism. Communications between the interrogator and the vulnerable device are enabled if the interrogator requests have been authenticated, thus preventing unauthorized requests from reaching the vulnerable device.

### CROSS REFERENCE TO RELATED APPLICATION

This application claims priority of provisional patent application Ser. No. 61/102,677, filed on Oct. 3, 2008, the entire disclosure of which is incorporated herein by reference.

### FIELD OF THE INVENTION

The invention relates generally to the field of Radio Frequency Identification (RFID) systems, and more specifically, to the use of zero-power, energy-harvesting computational modules to provide secure and reprogrammable wireless communications with devices comprising integrated circuits (ICs), including active implantable medical devices, electronic lock and key systems, credit cards, access cards, identification cards and passports.

### BACKGROUND OF THE INVENTION

Radio Frequency Identification (RFID) devices may be categorized as active or passive. An active RFID device contains a power source, such as a direct current battery, and can autonomously transmit signals. A passive RFID device requires no internal power supply, and is instead powered by the extremely small electrical current induced in the antenna by an incoming radio frequency (RF) signal from a remote reader or interrogator.

A block diagram of a typical prior art Zero-Power, Energy-Harvesting RFID unit is shown in FIG. 1. RFID unit 100 may include integrated circuits, such as a transceiver 120, memory 130 and a controller 140, and an antenna 150. As is known in the art, zero-power or passive RFIDs do not actively transmit radio signals. Instead, they modulate the impedance of their antenna using a transistor, which causes a change in the amount of energy reflected back to the RFID reader. This modulated reflection is typically called backscatter radiation. By way of example, U.S. Patent Application Publication No. US 2008/0143192, by Alanson P. Sample and Joshua R. Smith, discloses systems and methods for dynamically harvesting power from a radio frequency signal using a voltage doubler circuit.

Prior art RFID devices, such as RFID unit 100, typically have limited computational power. As a result, prior art RFID devices have been used primarily as a substitute for bar codes, in applications such as asset management, product tracking, building security, mobile payments, and animal identification. For example, U.S. Patent Application Publication No. US 2008/0041930, by Joshua R. Smith and Dirk Haehnel, discloses the use of an energy-harvesting RFID device to receive and store configuration parameters for a computer, personal data assistant or cellular telephone connected to the RFID device.

Of particular relevance to the present invention are the applications of RFID devices to devices comprising integrated circuits that require secure communication to control access to valuable resources. The device that comprises an integrated circuit typically provides access to a valuable resource such as medical technology, particularly implantable medical devices, a secured area via electronic lock and key systems, such as those used to access motor vehicles and residential and commercial properties, or secured information and services. Illustrative examples of these applications are discussed below.

### Implantable Medical Devices

RFID technology has been applied in the fields of healthcare and medical technology, and in particular, to medical devices that can be implanted in the human body. As defined by ISO 13485, the Quality Management Standard for Medical Devices, an implantable medical device (IMD) is a manufactured product that is partially or totally inserted into the human body or a natural orifice and is expected to stay there for thirty days or more. Surgical or medical procedures are used to insert or apply implantable medical devices, and surgical or medical procedures must be used to remove them.

IMDs may be classified as active or passive. An active IMD uses electrical energy or other sources of power to function, while a passive IMD does not. Examples of passive IMDs include artificial joints and artificial valves. Active IMDs may be used to treat diseases or injuries, or to replace or supplement a physiological function. Examples of active implantable medical devices include muscle stimulators, drug delivery systems, neurological stimulators, and cardiac rhythm management (CRM) devices such as implantable pacemakers and implantable cardioverter defibrillators (ICDs).

Active IMDs rely on integrated internal batteries, internal energy-harvesting systems, or external power sources to perform their tasks. If powered internally, active IMDs may use long-lasting batteries that can function for an average of five to seven years. These batteries are typically hard-wired to the IMD during manufacturing, before the IMD is hermetically sealed.

Replacing the battery, therefore, may also necessitate replacing the IMD, requiring surgery and an attendant risk to the patient. Further, although rare, batteries have been known to leak toxic substances, presenting yet another risk to the patient. Active IMDs may also be powered externally by a direct electrical or pneumatic linkage or a radio frequency (RF) link. These externally-powered devices, however, typically require a source of backup power as a safety precaution. An example of an externally-powered IMD is a left ventricular assist device (LVAD), which typically requires more power than can be provided by internal batteries.

In addition to their primary functions, the latest IMDs also support remote identification, monitoring, and control via standard telemetry systems. For example, many devices report measured data to healthcare providers and/or to patients, and may also allow authorized users to upgrade the IMD's firmware and software applications or to modify the IMD's settings, such as the IMD's therapy settings. For example, U.S. Pat. No. 7,177,699, issued to Willa Fabian, et al., discloses an implanted medical device that provides patient data to a home monitoring system and a remote monitoring system via standard telemetry systems, home network systems, wireless local area networks (WLAN), the Internet or cellular networks. Performing these additional monitoring and control functions, however, may further drain the IMD's battery and shorten its effective lifetime. While the use of a secondary battery for auxiliary purposes is known, this approach entails many of the same risks associated with primary batteries. Another approach to powering an IMD is the use of rechargeable batteries, as described in U.S. Pat. No. 6,798,716, issued to Arthur Charych. Rechargeable batteries, however, tend to be more expensive than non-rechargeable batteries, and may require a backup power source. Rechargeable batteries are also historically less predictable and reliable than single-use batteries for reasons such as heat and gas emissions.

Systems and methods have been developed to reduce unnecessary use of the IMD's battery and minimize power consumption. For example, IMDs have been designed to enter high energy consumption modes only when necessary. Some IMDs contain dual clocks for separate on-demand, high-frequency components and continual, low-frequency components.

Alternatively, or in addition, an IMD may be coupled with a passively-powered radio-frequency identification (RFID) device having an integrated circuit for storing and processing information, and an antenna for receiving and transmitting signals. To date, passive or zero-power, energy-harvesting RFIDs have been used extensively for identification, such as responding to requests for fixed identification (ID) numbers, or for supplying stored data on request.

Examples of passive or zero-power RFIDs used in conjunction with IMDs include U.S. Pat. No. 7,240,833, issued to Paul E. Zarembo, which discloses a system and method for managing information related to the manufacture of an IMD by storing and updating information in an RFID unit that is packaged with the IMD, and transferring the information from the RFID unit to other devices upon request from an external RFID interrogator or reader. U.S. Pat. No. 7,125,382, issued to Peter Zhou et al., discloses a bio-sensor system that utilizes RFID technology and includes a remote reader or interrogator in communication with an implantable passively-powered on-chip transponder. The remote reader or interrogator is configured to remotely receive identification information and data representative of a patient's physiological measurement transmitted by the on-chip transponder upon request from the remote reader or interrogator. U.S. Patent Application No. 2006/0212096, by Robert Stevenson, discloses an RFID system for use with an IMD, where an RFID tag implanted with the IMD may store information about the IMD, including the manufacturer, model number, and serial number. Note that in these examples, the function of the RFID is primarily one of identification, because historically, zero-powered RFIDs have limited computational power.

While it is important to limit the intentional use of the IMD's battery to preserve its useful life, it is perhaps just as important to prevent accidental or malicious draining of the IMD's primary battery. For example, because the newest IMDs can communicate with home networks and the Internet, they are susceptible to unauthorized manipulation of their settings and to denial-of-service attacks. A denial-of-service (DoS) attack is an attempt to make a resource unavailable, such as by saturating the resource with requests until the resource cannot respond to legitimate requests or responds so slowly as to become effectively unresponsive. For example, if a medical microcontroller, such as one known in the art, transfers 1 Mbyte of data at 500 Kbits/second every day for ten years, it is estimated that it would consume only about 10% of the total capacity of the device battery (1 Mbyte of data=8,000 Kbits of data, 8,000 Kbits at 500 Kbits/second=16 seconds/data transfer, 1 data transfer/day for 10 years=3,650 data transfers). However, if a malicious or accidental attack were to cause spurious wakeups, resulting in as many as 5,400 data transfers a day (86,400 seconds/day, at 16 seconds per transfer), the same battery could completely discharge in as little as seven days.

Traditional approaches to security, such as the use of passwords or a cryptographic key, are known, but may hinder treatment in an emergency setting if the password or cryptographic key is unavailable. An example of an attempt to address this issue is U.S. Pat. No. 6,880,085, issued to Ronald A. Balczewski and Karen Lent, which discloses a security system for programmable medical devices in which at least some features are only enabled if a proper password is provided.

Another risk to the availability of an IMD is excessive power consumption by mechanisms other than those required to provide the device's primary functions. For example, strong security mechanisms, such as public key cryptography, can be expensive in terms of both computational time and energy consumption. In addition, while strong cryptography for high frequency (HF) and low frequency (LF) RFID devices is well known, strong cryptography for ultra-high frequency (UHF) RFID devices has not been widely available due in part to their limited computation capabilities. UHF RFID devices are extremely resource-limited as compared to HF and LF devices, and the longer reading range available for UHF devices makes them more vulnerable to security attacks.

There is a need in the art, then, for systems and methods of improving communications with IMDs, and particularly for improving security and privacy for IMDs without draining the IMD's battery. Ideally, such zero-power systems and methods would prevent or deter malicious or accidental attacks on the IMD's firmware, software applications, settings, stored data, and power. In addition, communications with the IMD must be available in an emergency situation.

### Electronic Lock and Key Systems

Prior art electronic lock and key systems employ many different technologies, but in general, all use credentials, such as a magnetic card, smart card, proximity card, and/or a Personal Identification Number (PIN), to actuate the lock and permit access to a secured resource. Magnetic cards include a magnetic strip with an embedded code, and the lock is opened when the code matches a predefined criteria or algorithm. The surface of the magnetic strip, however, can be easily damaged and may become unusable. Smart cards that require contact with a surface, such as those that are swiped through a card reader, are also susceptible to physical damage.

RFID technology provides a partial solution to the problem of physical damage. For example, proximity cards may include a passive RFID tag, which is less susceptible to wear and tear than a magnetic strip, and may be used to gain entry to a building, laboratory, hotel room, or office suite. Remote keyless entry systems for automobiles are also well known. By way of example, the widely used KeeLoq® remote keyless entry (RKE) authentication system (Microchip Technology Inc., Chandler, Ariz.) consists of a receiver in the vulnerable object to be secured, such as a car door, and incorporates an active RFID transponder embedded in the remote control, such as the automobile key. The remote control sends transmissions to the receiver to control access to the vulnerable object. The KeeLoq® RKE systems use two types of keys. The first key is a device key that is unique to each remote control and is shared by the transmitter in the remote control and the receiver in the vulnerable device. The second key is a manufacturer key that is believed to be identical for all receivers for a specific manufacturer, and is used primarily used for deriving the device keys.

These RFID-based remote keyless entry systems, however, may not be entirely secure. There have been reports that some of these devices have had their security algorithms compromised by eavesdroppers. For example, researchers have used differential power analysis (DPA) attacks on KeeLoq® RKE systems to attack both the transmitters and receivers, as described in the paper, “On the Power of Power Analysis in the Real World: A Complete Break of the KeeLoq Code Hopping Scheme,” by Thomas Eisenbarth, Timo Kasper, Amir Moradi, Christof Paar, Mahmoud Salmasizadeh, Mohammad T. Manzuri Shalmani. (28th International Cryptology Conference—CRYPTO 2008. Santa Barbara, Calif., USA. Aug. 17-21, 2008, available at http://www.crypto.rub.de/imperia/md/content/texte/publications/conferences/crypto2008_keeloq. pdf and http://www.springerlink.com/content/b83338g657112111/. Researchers have also employed simple power analysis (SPA) methods to reveal the secret keys used in KeeLoq® RKE systems, as described in the paper, “Breaking KeeLoq in a Flash: On Extracting Keys at Lightning Speed,” by Markus Kasper, Timo Kasper, Amir Moradi, Christof Paar. (2nd International Conference on Cryptology in Africa, Progress in Cryptology—AFRICACRYPT 2009, Gammarth, Tunisia, Jun. 21-25, 2009, available at http://www.crypto.rub.de/imperia/md/content/texte/publications/conferences/africacrypt2009_ke eloq.pdf and http://www.springerlink.com/content/e44438x977808257/.

### Access to Secured Information and Services

RFID credit cards are known in the art, and their security concerns have been examined. See, e.g., Heydt-Benjamin, T. S., Bailey, D. V., Fu, K., Juels, A., and O'Hare, T., Vulnerabilities in First-Generation RFID-enabled Credit Cards, http://www.cs.umass.edu/˜kevinfu/papers/RFID-CC-manuscript.pdf, accessed Jul. 29, 2009. In addition, in Reverse-Engineering a Cryptographic RFID Tag, by Karsten Nohl, David Evans, Starbug, and Henry Plotz (USENIX Security. August 2008, available at http://www.cs.virginia.edu/˜evans/pubs/usenix08/usenix08.pdf), the authors revealed the cipher implemented on the NXP Mifare Classic RFID tags. Because NXP hardcoded their algorithm, it could not be modified or repaired, and the manufacturer reportedly advised that the millions, perhaps billions, of cards in circulation be physically replaced.

In general, devices comprising integrated circuits can be associated with financial information and services, access to transportation services, such as toll road payments and subway fares, and passports and other forms of identification, providing numerous potential opportunities for identity theft and theft of services. These RFID credit cards are often treated as “throw-away,” because they cannot be reprogrammed; the only way to modify the algorithms, and thus the way the card behaves, is to issue the user a completely new card. As with the RKE systems, all the cards in a particular system would need to be replaced if a security flaw were discovered in the card's algorithm.

There is need for upgradable zero-power security for communication with vulnerable devices comprising integrated circuits. The algorithmic flaws in the security algorithms, such as those described in the above-referenced papers, have compromised the integrity of some existing systems. Because these systems cannot be reprogrammed, new physical keys must be issued to replace the old keys. In the context of security systems, reprogramming means more than merely changing the value of stored data, such as a key, password or the value of a register. To ensure security, the executable programs on the zero-power devices themselves must be replaced, a capability that is not available in currently available systems. In addition to the security issues, swapping old keys out for new keys is not only expensive, but logistically challenging. There is a need in the art then, for a more secure, and reprogrammable, remote keyless entry system.

### SUMMARY OF THE INVENTION

The present invention provides systems and methods for utilizing zero-power, energy-harvesting computational modules to provide secure and reprogrammable wireless communications with devices comprising integrated circuits (ICs), including active implantable medical devices, electronic lock and key systems, credit cards, access cards, identification cards and passports.

In preferred embodiments, the present invention provides a system comprising a device comprising at least one integrated circuit, wherein the device is vulnerable to unauthenticated access; an antenna, and; a zero-power, energy-harvesting reprogrammable computational module configured to communicate with the antenna to receive radio frequency signals and to communicate with the device, wherein the computational module is powered by a corresponding radio frequency signal and verifies an authentication request sent in the corresponding radio frequency signal using a secure challenge-response cryptographic function. Typically, the system includes an interrogator configured to transmit the corresponding radio frequency signal. In certain preferred embodiments, the device further comprises a battery. The antenna is optionally integrated with the computational module. In general, the device comprises non-volatile memory. In preferred embodiments, the device comprises a microcontroller having a data bus and non-volatile memory.

In certain embodiments, the system also includes a UHF transmitter or transceiver in communication with the device; and a UHF antenna in communication with the UHF transmitter or transceiver. Typically, the computational module comprises a microcontroller, and is hard-wired to the enabled device. In other embodiments, the computational module communicates wirelessly with the device.

In preferred embodiments, the computational module and the interrogator communicate using a RFID protocol. In various embodiments, the RFID protocol is an Electronic Product Code (EPC) Class 1 Generation 1 protocol, EPC Class 1 Generation 2 protocol, ISO/IEC 7816, ISO/IEC 14443 or ISO/IEC 18092. In various embodiments, the radio frequency signal can be an ultra high frequency (UHF) signal, a high frequency (HF) signal, a low frequency (LF) signal, or a medical implant communications service (MICS) signal.

In some preferred embodiments, the device is an implantable medical device. In other preferred embodiments, the system is uses in a keyless access system, and can be incorporated in an automobile key, a key fob, a building access card or a room access card. In other preferred embodiments, the system is incorporated in a contactless smart card. In further preferred embodiments, the system is incorporated in a travel document, a driver's license, a personal identity verification card, a medical identity card or an employee identity card.

In certain preferred embodiments, the interrogator is authenticated using a symmetric cryptographic function. In other embodiments, the interrogator is authenticated using an asymmetric cryptographic function. In certain preferred embodiments, the system performs the steps of sending a unique identifier to the interrogator, sending a nonce to the interrogator, computing a key value using the unique identifier and the nonce, computing a key value using the unique identifier, comparing the computed key value to a key value received from the interrogator, and sending an authentication status to the interrogator. Typically, the key value is computed using a cryptographic function. In certain preferred embodiments, the key value is computed using an Advanced Encryption Standard (AES) algorithm.

In other aspects, preferred embodiments of the present invention provide a method of secure radio frequency communication with a vulnerable device, comprising the steps of providing a system comprising a device comprising at least one integrated circuit, wherein the device is vulnerable to unauthenticated access; an antenna, and; a zero-power, energy-harvesting reprogrammable computational module configured to communicate with the antenna to receive radio frequency signals and to communicate with the device, wherein the computational module is powered by a corresponding radio frequency signal and authenticates the source of the corresponding radio frequency signal using a cryptographic function; using the system to receive a corresponding radio frequency signal from an interrogator, storing the energy in the received corresponding radio frequency signal; authenticating the interrogator using an encrypted challenge-response authentication mechanism between the zero-power, energy-harvesting reprogrammable computational module and the interrogator; and enabling communication between the interrogator and the device if the interrogator is authenticated.

In certain preferred embodiments, the device further comprises a battery. The antenna is optionally integrated with the computational module. In general, the device comprises non-volatile memory. In preferred embodiments, the device comprises a microcontroller having a data bus and non-volatile memory.

In certain embodiments, the system also includes a UHF transmitter or transceiver in communication with the device; and a UHF antenna in communication with the UHF transmitter or transceiver. Typically, the computational module comprises a microcontroller, and is hard-wired to the enabled device. In other embodiments, the computational module communicates wirelessly with the device.

In preferred embodiments, the computational module and the interrogator communicate using a RFID protocol. In various embodiments, the RFID protocol is an Electronic Product Code (EPC) Class 1 Generation 1 protocol, EPC Class 1 Generation 2 protocol, ISO/IEC 7816, ISO/IEC 14443 or ISO/IEC 18092. In various embodiments, the radio frequency signal can be an ultra high frequency (UHF) signal, a high frequency (HF) signal, a low frequency (LF) signal, or a medical implant communications service (MICS) signal.

In some preferred embodiments, the device is an implantable medical device. In other preferred embodiments, the system is uses in a keyless access system, and can be incorporated in an automobile key, a key fob, a building access card or a room access card. In other preferred embodiments, the system is incorporated in a contactless smart card. In further preferred embodiments, the system is incorporated in a travel document, a driver's license, a personal identity verification card, a medical identity card or an employee identity card.

In other preferred embodiments, present invention provides a method for communicating with an implantable medical device (IMD) comprising the steps of receiving a radio frequency (RF) signal from an interrogator, where the RF signal comprises a command for the IMD; harvesting energy from the RF signal; and sending the command to the IMD. In preferred embodiments, the method can further comprise the steps of storing the energy harvested from the RF signal, and determining if there is sufficient harvested energy before sending the command to the IMD. The method can also include one or more of the steps of receiving a reply from the IMD, performing a computation before sending the response to the interrogator, and sending a response to the interrogator.

In further preferred embodiments, present invention provides a method for securely communicating with an implantable medical device (IMD) comprising the steps of receiving an RF signal comprising an authentication request from an interrogator; authenticating the interrogator using energy harvested from the RF signal; and permitting access to the IMD if the interrogator is authenticated.

In certain preferred embodiments, the interrogator is authenticated using a symmetric cryptographic function. In other embodiments, the interrogator is authenticated using an asymmetric cryptographic function. In certain preferred embodiments, the system performs the steps of sending a unique identifier to the interrogator, sending a nonce to the interrogator, computing a key value using the unique identifier and the nonce, computing a key value using the unique identifier, comparing the computed key value to a key value received from the interrogator, and sending an authentication status to the interrogator. Typically, the key value is computed using a cryptographic function. In certain preferred embodiments, the key value is computed using an Advanced Encryption Standard (AES) algorithm. In preferred embodiments, method includes the step of receiving a command for the IMD.

In further preferred embodiments, present invention provides a system comprising an electronic lock comprising an interrogator; a zero-power, energy-harvesting computational module configured to communicate with the electronic lock; and where the interrogator for wirelessly transmits a radio frequency (RF) signal to the computational module, where the computational module is powered by the incoming RF signal, wherein the computational module authenticates the interrogator using a secure challenge-response cryptographic function and the electronic lock is opened if the interrogator is authenticated.

In certain embodiments, the system also includes a UHF transmitter or transceiver in communication with the device; and a UHF antenna in communication with the UHF transmitter or transceiver. Typically, the computational module comprises a microcontroller, and is hard-wired to the enabled device. In other embodiments, the computational module communicates wirelessly with the device.

In preferred embodiments, the computational module and the interrogator communicate using a RFID protocol. In various embodiments, the RFID protocol is an Electronic Product Code (EPC) Class 1 Generation 1 protocol, EPC Class 1 Generation 2 protocol, ISO/IEC 7816, ISO/IEC 14443 or ISO/IEC 18092. In various embodiments, the radio frequency signal can be an ultra high frequency (UHF) signal, a high frequency (HF) signal, a low frequency (LF) signal, or a medical implant communications service (MICS) signal.

In certain preferred embodiments, the interrogator is authenticated using a symmetric cryptographic function. In other embodiments, the interrogator is authenticated using an asymmetric cryptographic function. In certain preferred embodiments, the system performs the steps of sending a unique identifier to the interrogator, sending a nonce to the interrogator, computing a key value using the unique identifier and the nonce, computing a key value using the unique identifier, comparing the computed key value to a key value received from the interrogator, and sending an authentication status to the interrogator. Typically, the key value is computed using a cryptographic function. In certain preferred embodiments, the key value is computed using an Advanced Encryption Standard (AES) algorithm.

### DEFINITIONS

As used herein, the Advanced Encryption Standard (AES) is a symmetric key cipher that operates on blocks, or fixed length groups of bits. AES is also known as Rijndael.

A used herein, a Contactless Smart Card is a smart card that communicates with a reader through a radio frequency interface.

As used herein, the Data Encryption Standard (DES) is a symmetric key cipher that operates on blocks, or fixed length groups of bits.

As used herein, an ePassport is a travel document that contains an integrated circuit chip based on international standard ISO/IEC 14443 and that can securely store and communicate the ePassport holder's personal information to authorized reading devices.

As used herein, Flash Memory is non-volatile computer memory.

As used herein, a Hashing Algorithm is defined as an algorithm that, when applied to the information content of a variable length message, produces a fixed-length string called a hash value or hash.

As used herein, Ultra High Frequency (UHF) is the frequency band of electromagnetic waves between 300 MHz and 3 GHz.

As used herein, High Frequency (HF) is the frequency band of electromagnetic waves between 3 MHz and 30 MHz.

As used herein, Low Frequency (LF) is the frequency band of electromagnetic waves between 30 kHz and 300 kHz.

As used herein, ISO/IEC 7816 is an international standard for integrated circuit cards (i.e., Smart Cards) with contacts as well as the command set for all Smart Cards.

As used herein, ISO/IEC 14443 is ISO/IEC standard “Identification Cards—Contactless Integrated Circuit(s) Cards—Proximity Cards.” The international standard for contactless smart chips and cards that operate (i.e., can be read from or written to) at a distance of less than 10 centimeters (4 inches). This standard operates at 13.56 MHz.

As used herein, the Medical Implant Communications Service (MICS) is an ultra-low power, unlicensed, mobile radio service for transmitting data in support of diagnostic or therapeutic functions associated with implanted medical devices, having a frequency band of 402-405 MHz. The MICS permits individuals and medical practitioners to utilize ultra-low power medical implant devices, such as cardiac pacemakers and defibrillators, without causing interference to other users of the electromagnetic radio spectrum.

As used herein, Near Field Communication (NFC) is a short-range wireless standard (ISO/IEC 18092) that uses magnetic field induction to enable communication between devices when they are brought close together (within 10-20 centimeters or 4-8 inches). NFC technology is compatible with ISO/IEC 14443-based technology.

As used herein, a Nonce, or Cryptographic Nonce, is a random or pseudo-random number used in an authentication protocol. Nonce means a “number used once.”

As used herein, Non-volatile Memory is memory that holds data even after its power source is removed.

As used herein, a PIV Card (Personal Identity Verification Card) is a dual-interface (contact and contactless) Smart Card issued to all U.S. Executive Branch Federal employees and contractors and that will be used for both physical and logical access.

As used herein, Public Key Cryptography is a form of cryptography that uses a pair of cryptographic keys, a public key and a private key. A message encrypted with the public key can only be decrypted with the private key. Public key cryptography is also known as asymmetric cryptography.

As used herein, Random Access Memory (RAM) is a form of computer data storage, in which stored data can be accessed in any order and in a constant time, regardless of its physical location in storage and its relationship to contiguous pieces of data.

As used herein, RC5 is a symmetric key cipher that operates on blocks, or fixed length groups of bits.

As used herein, a Reader or an Interrogator is any device that communicates information or assists in communications from a card, token, implantable medical device, or other device and transmits the information to a host such as a control panel/processor or database for further action.

As used herein, Secret Key Cryptography is a form of cryptography that uses a single secret key for both encryption and decryption.

As used herein, a Smart Card is a device that includes an embedded secure integrated circuit that can be either a secure microcontroller or equivalent intelligence with internal memory or a secure memory chip alone. The card connects to a reader with direct physical contact or with a remote contactless radio frequency interface. With an embedded microcontroller, smart cards have the ability to securely store large amounts of data, carry out their own on-card functions (e.g., encryption and mutual authentication) and interact intelligently with a smart card reader. Smart card technology conforms to international standards (ISO/IEC 7816 and ISO/IEC 14443) and is available in a variety of form factors, including plastic cards, subscriber identification modules (SIMs) used in GSM mobile phones, and USB-based tokens.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a block diagram of a typical prior art RFID unit;

FIG. 2 is a block diagram of a preferred embodiment of the zero-power, energy-harvesting computational module incorporated into a device having an integrated circuit, and specifically, a device having a controller and memory;

FIG. 3 is a block diagram of a preferred embodiment of the zero-power secure communications system of the present invention, showing the use of a zero-power, energy-harvesting computational module incorporated with an implantable medical device;

FIGS. 4A and 4B provide a functional flow chart of the processing performed by the zero-power, energy-harvesting computational module in the embodiment shown in FIG. 3;

FIG. 5 provides a functional flow chart of the secure challenge-response protocol of the embodiment shown in FIG. 3;

FIG. 6 is a block diagram of a preferred embodiment of the zero-power secure communications system of the present invention, showing the use of a zero-power, energy-harvesting module incorporated into an electronic lock and key system;

FIGS. 7A and 7B provide a functional flow chart of the processing performed by the zero-power, energy-harvesting computational module in the embodiment shown in FIG. 6; and

FIG. 8 provides a functional flow chart of the secure challenge-response protocol of the embodiment shown in FIG. 6.

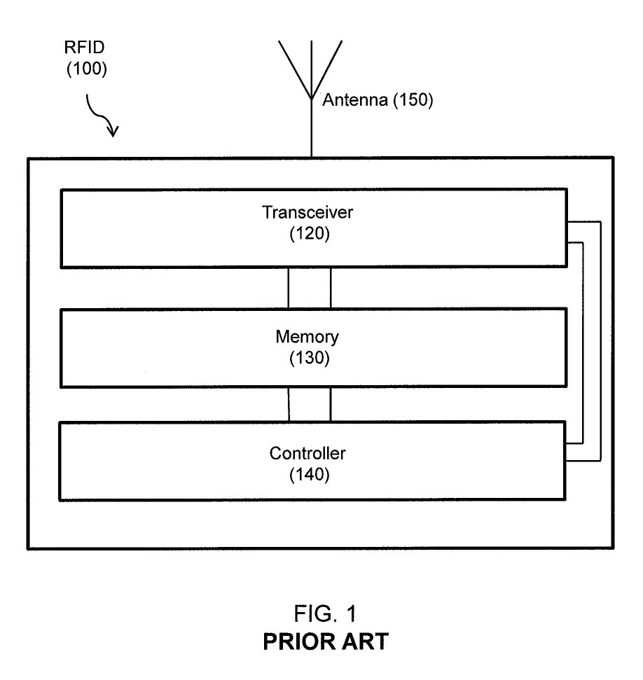

### DETAILED DESCRIPTION OF PREFERRED EMBODIMENTS

The invention provides systems and methods for utilizing zero-power, energy-harvesting computational modules to provide secure and reprogrammable wireless communications with devices comprising integrated circuits (ICs), including active implantable medical devices, electronic lock and key systems, credit cards, access cards, identification cards and passports.

### Zero-Power, Energy-Harvesting Computation Module

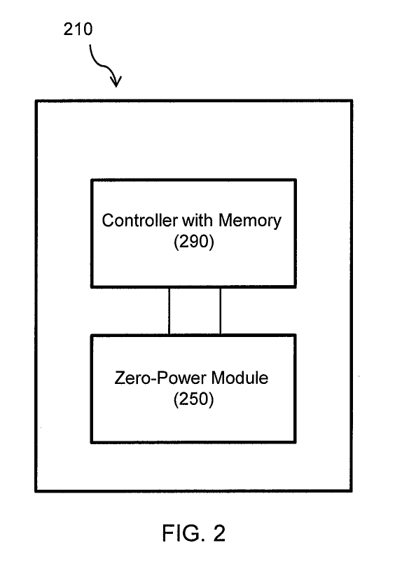

While the present invention is not limited to a particular zero-power, energy-harvesting computational module, a preferred embodiment of the invention incorporates Intel's Wireless Identification and Sensing Platform (WISP). WISP is a battery-free, microcontroller-based UHF device that implements RFID protocols in software. In a preferred embodiment, WISP uses a TI MSP430 programmable microcontroller that is powered and read by a standards-compliant ultra high frequency (UHF) RFID reader running the Electronic Product Code (EPC) Class 1 Generation 1 protocol. This protocol defines physical and logical requirements for a passive-backscatter, interrogator-talks-first (ITF), radio frequency identification (RFID) system. The TI MSP430 is an ultra low power microcontroller that provides general-purpose computational capabilities and runs at a maximum speed of 8 MHz. In a preferred embodiment, the WISP includes approximately 8 Kbytes of flash memory and 256 bytes of random access memory (RAM). The present invention is not limited to this particular version of the WISP, and use of the next-generation WISP, which supports the Electronic Product Code (EPC) Class 1 Generation 2 protocol, is also suitable. The present invention is also not limited to computational modules incorporating microcontrollers. Note also that the present invention is not limited to using UHF signals, and the use of LF, HF, and MICS signals is also contemplated. Zero-Power, Energy-Harvesting Computational Module Incorporated with a Device Comprising an Integrated Circuit FIG. 2 is a block diagram of a preferred embodiment of the zero-power, energy-harvesting computational module incorporated into a device having an integrated circuit. As shown in FIG. 2, device comprising an integrated circuit 210 includes a Controller with Read/Write Memory 290 and a Zero-Power, Energy-Harvesting Computational Module 250. In a preferred embodiment, Zero-Power, Energy-Harvesting Computational Module 250 is hard-wired to Controller with Read/Write Memory 290. In alternate embodiments, Zero-Power, Energy Harvesting Computational Module 250 may communicate wirelessly with Controller with Read/Write Memory 290, reducing the possibility of failures associated with faulty wiring. In preferred embodiments, device 210 may be an active implantable medical device, an electronic key in an electronic lock and key system, a credit cards, an access card, an identification card or a passport, although the invention is not limited to these embodiments.

### Zero-Power Communications System Implantable Medical Device (IMD)

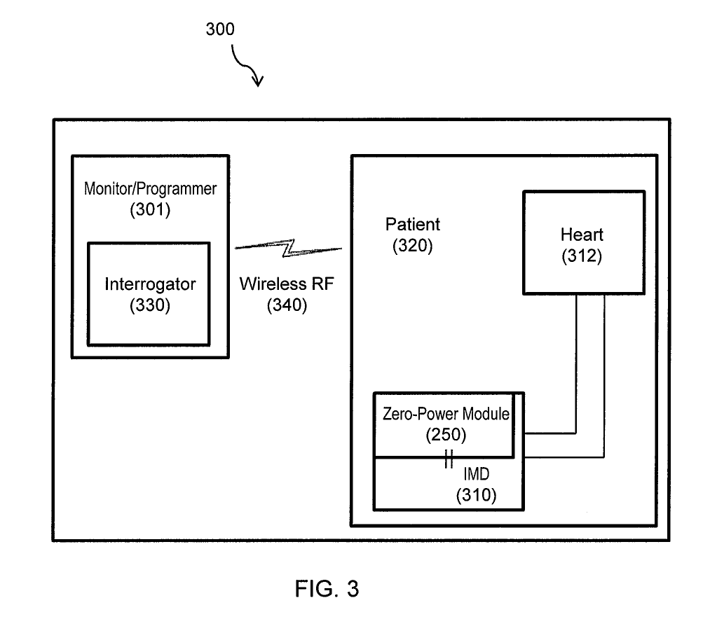

FIG. 3 is a block diagram of a preferred embodiment of the zero-power secure communications system of the present invention, as incorporated with an implantable medical device (IMD). With reference to FIG. 3, by way of example, secure communications system 300 includes an IMD 310, such as a cardiac resynchronization therapy (CRT) device, which has been implanted in a patient 320. As is known in the art, IMD 310 may be connected by one or more leads 311 to the patient's heart 312. The invention, however, is not limited to a particular type of IMD or physiological function, and the use of other types of IMDs or embedded devices, including but not limited to muscle stimulators and drug delivery systems, are within the scope of the invention.

With further reference to FIG. 3, in a preferred embodiment, IMD 310 includes a Zero-Power, Energy-Harvesting Computational Module 250, which is hard-wired to IMD 310. In alternate embodiments, Zero-Power, Energy Harvesting Computational Module 250 may communicate wirelessly with IMD 310, reducing the possibility of failures associated with faulty wiring.

Secure communications system 300 also includes Interrogator 330, also known as a reader or programmer, which may be included in a Remote Monitor 301. Interrogator 330 provides incoming radio frequency (RF) energy 340, which is converted to direct current (DC) power by the energy-harvesting circuitry of Zero-Power, Energy-Harvesting Computational Module 250.

### Zero-power RFID Processing—Implantable Medical Device

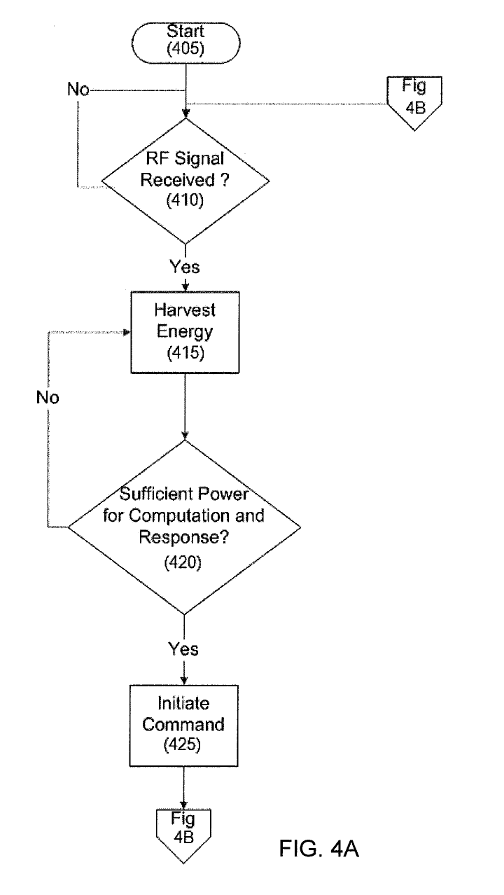

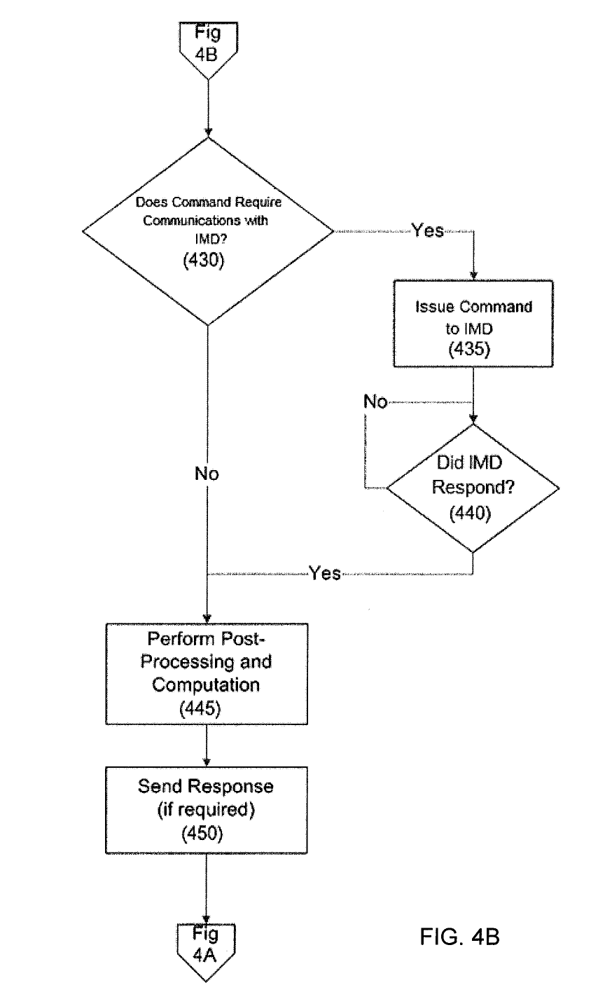

FIGS. 4A and 4B provide a functional flow chart of the processing performed by the Zero-Power, Energy-Harvesting Computational Module 250 in a preferred embodiment of the invention shown in FIG. 3. With reference to FIG. 4A, at step 410, the Zero-Power, Energy-Harvesting Computational Module 250 determines whether an RF signal has been received from an external device such as an Interrogator 330. In alternate embodiments, Zero-Power, Energy-Harvesting Computational Module 250 may also receive a signal from another IMD. If an RF signal is detected, Zero-Power, Energy-Harvesting Computational Module 250 harvests energy from the external device, specifically by rectifying incoming RF energy into DC voltage, in step 415. In step 420, if Zero-Power, Energy-Harvesting Computational Module 250 determines that there is sufficient harvested energy to perform computation and provide a response, Zero-Power, Energy-Harvesting Computational Module 250 will initiate the command, as in step 425. In a preferred embodiment, Zero-Power, Energy-Harvesting Computational Module 250 waits for a threshold value of approximately 3 V (Volts) to build up on an internal capacitor before powering the on-board microcontroller. In alternate embodiments, the threshold value is dependent upon expected workload. For example, a computation that requires writing to flash memory requires more energy than a computation that does not require a write operation. In a preferred embodiment, the capacitor is a 10 μF (micro Farads) capacitor.

With reference to FIG. 4B, in step 430 Zero-Power, Energy-Harvesting Computational Module 250 determines if the command requires communication with the IMD. For example, a request from Interrogator 330 to change therapy data would require communication with the IMD, while a request from Interrogator 330 for data stored in Zero-Power, Energy-Harvesting Computational Module 250 would not require communication with the IMD. If communication with the IMD is required, in step 435 Zero-Power, Energy-Harvesting Computational Module 250 issues the command to the IMD. If the IMD responds, as shown in step 440, or if the command does not require communication with the IMD, Zero-Power, Energy-Harvesting Computational Module 250 performs post-processing and computations in step 445. In step 450, Zero-Power, Energy-Harvesting Computational Module 250 sends a radio response to the requesting device, if a response is required. In a preferred embodiment, IMD 310 and Zero-Power, Energy-Harvesting Computational Module 250 are physically wired together to provide a reliable communications channel. In alternate embodiments, Zero-Power, Energy-Harvesting Computational Module 250 may communicate wirelessly with IMD 310. In additional embodiments, if the communications channel is less secure, and Zero-Power, Energy-Harvesting Communications Module 250 may resend the request to IMD 310 if IMD 310 does not respond.

Note that in an emergency situation, such as when an IMD detects a heart arrhythmia, Zero-Power, Energy-Harvesting Communications Module 250 may be bypassed, allowing direct communication with IMD 310.

### Secure Challenge-Response Authentication—Implantable Medical Device

In addition to requiring an external entity to provide the wireless power required to activate the Zero-Power, Energy-Harvesting Computational Module 250, the secure communications system of the present invention incorporates a secure challenge-response authentication mechanism to prevent unauthorized requests from accessing the IMD. Empirical studies have shown that symmetric cryptography, and specifically RC5, is feasible on microcontroller-based zero-power, energy-harvesting computational modules, such as the WISP of the preferred embodiments of the invention. Moreover, microcontroller-based zero-power, energy-harvesting computational modules allow for reprogramming to provide more flexible software updates. Such software updates provide a safe mechanism for maintaining the operation and the algorithms used by the zero-power, energy-harvesting computational modules. In contrast, purely hardware-based RFIDs, as known in the prior art, are inflexible should a design problem need to be corrected, as the device must be physically replaced.

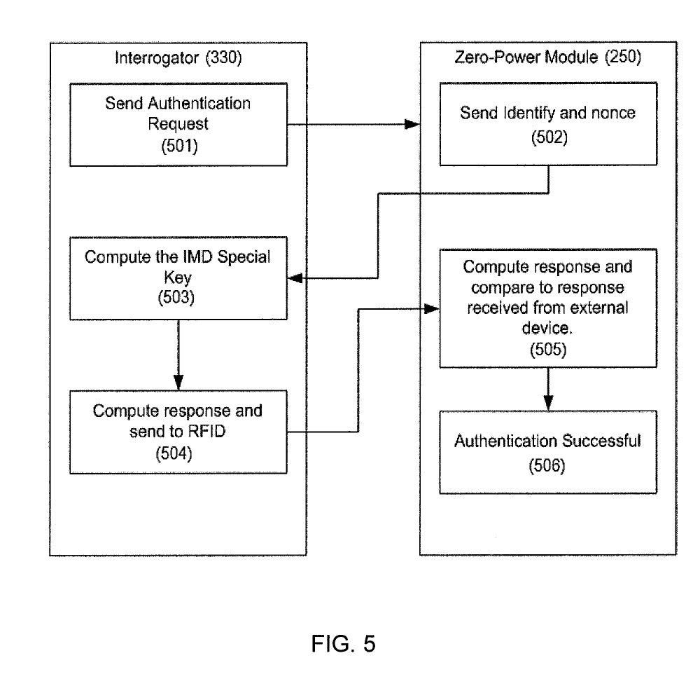

In a preferred embodiment, the secure challenge-response protocol of the invention is based on the RC5 block cipher, using 32-bit words, 12 rounds, and a 16-byte secret key (RC5-32/12/16). FIG. 5 is a functional flow chart of a preferred embodiment of the protocol of the invention. As shown in FIG. 5, an external device such as Interrogator 330 first transmits an authentication request in step 501 to the Zero-Power, Energy-Harvesting Computational Module 250, and the Zero-Power, Energy-Harvesting Computational Module 250 responds with its unique serial number or identity (I) and a nonce (N) in step 502. In step 503, Interrogator 330 computes the IMD-specific key (K) as:

K=ƒ(K_{m, I}),

where Kₘ is the master key, I is the unique identity of the IMD, and ƒ is any cryptographically strong pseudorandom function. In a preferred embodiment, function (ƒ) is the Advanced Encryption Standard (AES), although in alternate embodiments, DES or RC5 algorithms could be used. In a preferred embodiment, the value of Kₘ should be stored in a secure location in the external device.

With further reference to FIG. 5, in step 504 the Interrogator 230 computes the response (R) and sends it to the Zero-Power, Energy-Harvesting Computational Module 250, where R is:

R=RC5(K, N),

where RC5 is a block cipher algorithm. Note that the block cipher algorithm may be a symmetric or an asymmetric cipher.

In step 505, the Zero-Power, Energy-Harvesting Computational Module 250 also computes the response (R′), using the same function, and compares the computed response (R′) to the response received from the external device or Interrogator 330 (R). If the two response values match, as in step 506, the authentication is successful. In a preferred embodiment, the Zero-Power, Energy-Harvesting Computational Module 250 returns a “not authenticated” status to Interrogator 330 if the two response values do not match and an “authenticated” status to Interrogator 330 if the two response values match. Once authenticated, communications between the Interrogator 330 and the Zero-Power, Energy-Harvesting Computational Module 250 may proceed as described above and shown in FIGS. 4A and 4B.

### Zero-Power Communications System—Electronic Lock and Key System

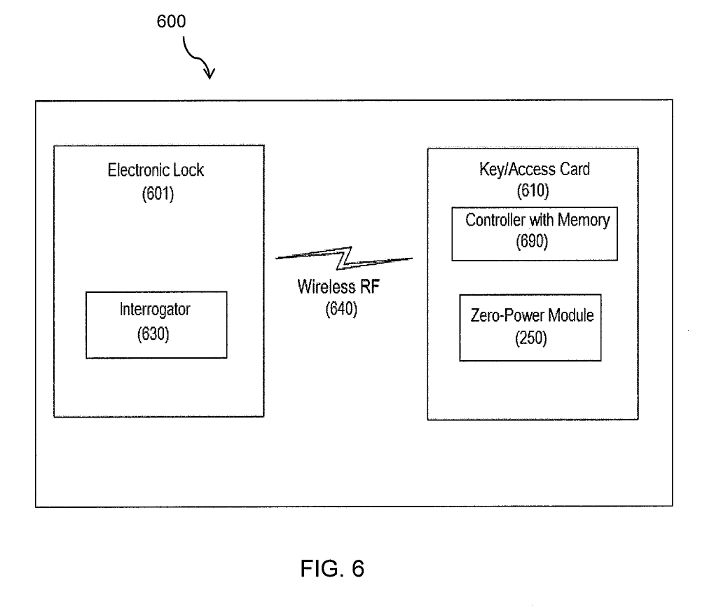

FIG. 6 is a block diagram of a preferred embodiment of the zero-power secure communications system of the present invention, as incorporated into an electronic lock and key system. With reference to FIG. 6, secure communications system 600 includes a Key or Access Card 610, which incorporates Zero-Power, Energy-Harvesting Computational Module 250 and a Controller with Read/Write Memory 690. In a preferred embodiment, Zero-Power, Energy-Harvesting Computational Module 250 is hard-wired to Controller with Read/Write Memory 690. In alternate embodiments, Zero-Power, Energy Harvesting Computational Module 250 may communicate wirelessly with Controller with Read/Write Memory 690, reducing the possibility of failures associated with faulty wiring.

Secure communications system 600 also includes Interrogator 630, also known as a reader or programmer, which may be included in an Electronic Lock 601. Interrogator 630 provides incoming radio frequency (RF) energy 640, which is converted to direct current (DC) power by the energy-harvesting circuitry of Zero-Power, Energy-Harvesting Computational Module 250.

### Zero-Power RFID Processing—Electronic Lock and Key System

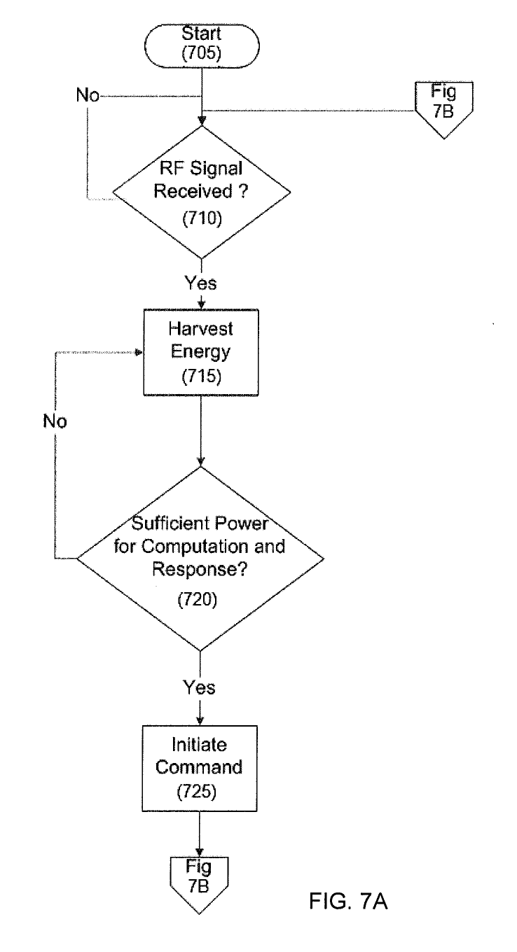

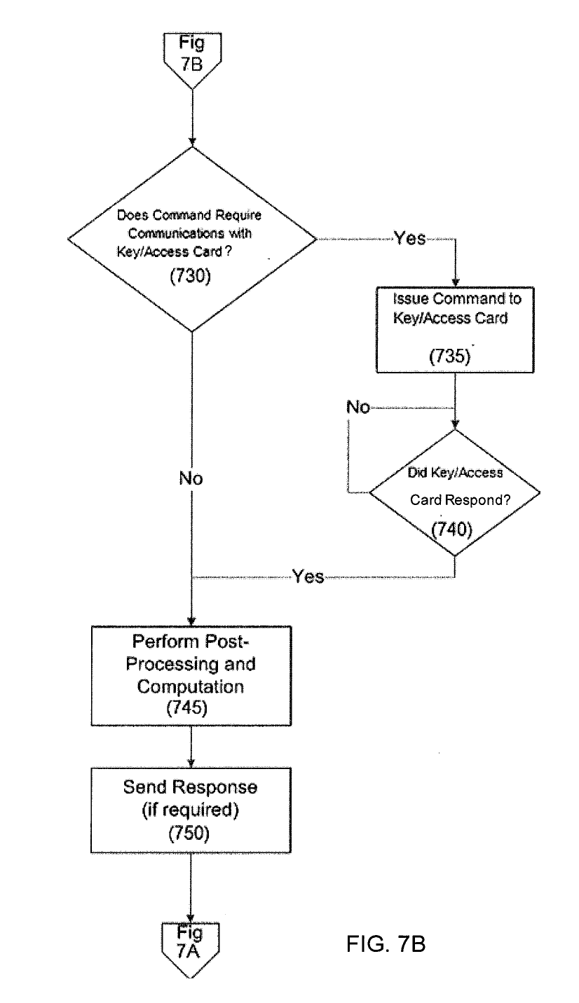

FIGS. 7A and 7B provide a functional flow chart of the processing performed by the Zero-Power, Energy-Harvesting Computational Module 250 in a preferred embodiment of the invention as shown in FIG. 6. With reference to FIG. 7A, at step 710, the Zero-Power, Energy-Harvesting Computational Module 250 determines whether an RF signal has been received from an external device such as an Interrogator 630. If an RF signal is detected, Zero-Power, Energy-Harvesting Computational Module 250 harvests energy from the external device, specifically by rectifying incoming RF energy into DC voltage, in step 715. In step 720, if Zero-Power, Energy-Harvesting Computational Module 250 determines that there is sufficient harvested energy to perform computation and provide a response, Zero-Power, Energy-Harvesting Computational Module 250 will initiate the command, as in step 725. In a preferred embodiment, Zero-Power, Energy-Harvesting Computational Module 250 waits for a threshold value of approximately 3 V (Volts) to build up on an internal capacitor before powering the on-board microcontroller. In alternate embodiments, the threshold value is dependent upon expected workload. For example, a computation that requires writing to flash memory requires more energy than a computation that does not require a write operation. In a preferred embodiment, the capacitor is a 10 μF (micro Farads) capacitor.

With reference to FIG. 7B, in step 730 Zero-Power, Energy-Harvesting Computational Module 250 determines if the command requires communication with the Controller with Read/Write Memory 690. For example, a request from Interrogator 630 to load a new cryptographic algorithm to the Key or Access Card 610 would require communication with the Controller with Read/Write Memory 690, while a request from Interrogator 630 for data stored in Zero-Power, Energy-Harvesting Computational Module 250 would not require communication with the Controller with Read/Write Memory 690. If communication with the Controller with Read/Write Memory 690 is required, in step 735 Zero-Power, Energy-Harvesting Computational Module 250 issues the command to the Controller with Read/Write Memory 690. If the Controller with Read/Write Memory 690 responds, as shown in step 740, or if the command does not require communication with the Controller with Read/Write Memory, Zero-Power, Energy-Harvesting Computational Module 250 performs post-processing and computations in step 745. In step 750, Zero-Power, Energy-Harvesting Computational Module 250 sends a radio response to the requesting device, if a response is required. In additional embodiments, if the communications channel is less secure, and Zero-Power, Energy-Harvesting Communications Module 250 may resend the request to Controller with Controller with Read/Write Memory 690 if Controller with Read/Write Memory does not respond.

### Secure Challenge-Response Authentication—Electronic Lock and Key System

In addition to requiring an external entity to provide the wireless power required to activate the Zero-Power, Energy-Harvesting Computational Module 250, the secure communications system of the present invention incorporates a secure challenge-response authentication mechanism to prevent unauthorized requests from accessing the Controller with Read/Write Memory 690 of Key or Access Card 610. As described above, empirical studies have shown that symmetric cryptography, and specifically RC5, is feasible on microcontroller-based zero-power, energy-harvesting computational modules, such as the WISP of the preferred embodiments of the invention. Moreover, microcontroller-based zero-power, energy-harvesting computational modules allow for more flexible software updates. Such updates provide a safe mechanism for maintaining the operation and the algorithms used by the zero-power, energy-harvesting computational modules. In contrast, purely hardware-based RFIDs, as known in the prior art, are inflexible should a design problem need to be corrected, as the Key or Access Card must be physically replaced.

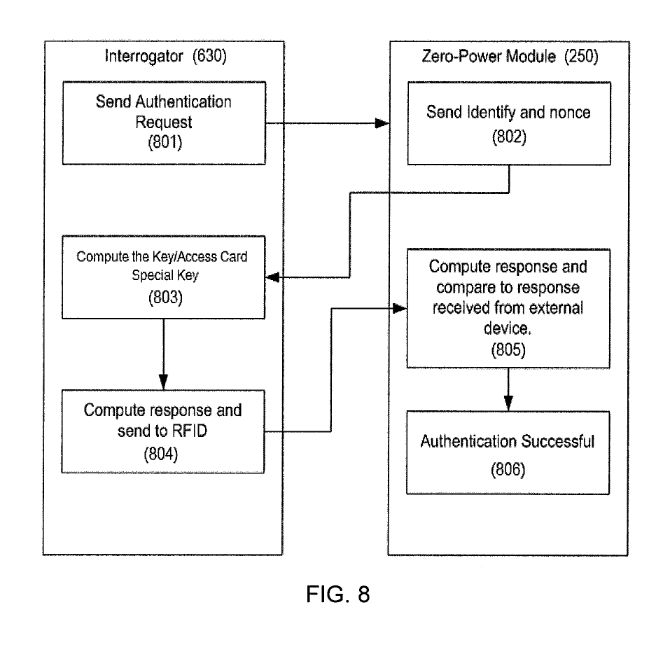

In a preferred embodiment, the secure challenge-response protocol of the invention is based on the RC5 block cipher, using 32-bit words, 12 rounds, and a 16-byte secret key (RC5-32/12/16). FIG. 8 is a functional flow chart of a preferred embodiment of the protocol of the invention. As shown in FIG. 6, an external device such as Interrogator 630 first transmits an authentication request in step 801 to the Zero-Power, Energy-Harvesting Computational Module 250, and the Zero-Power, Energy-Harvesting Computational Module 250 responds with its unique serial number or identity (I) and a nonce (N) in step 802. In step 803, Interrogator 630 computes the Key or Access Card-specific key (K) as:

K=ƒ(Kₘ, I),

where Kₘ is the master key, I is the unique identity of the Key or Access Card, and ƒ is any cryptographically strong pseudorandom function. In a preferred embodiment, function (ƒ) is the Advanced Encryption Standard (AES), although in alternate embodiments, DES or RC5 algorithms could be used. In a preferred embodiment, the value of Kₘ should be stored in a secure location in the external device.

With further reference to FIG. 8, in step 804 the Interrogator 630 computes the response (R) and sends it to the Zero-Power, Energy-Harvesting Computational Module 250, where R is:

R=RC5(K, N),

where RC5 is a block cipher algorithm. Note that the block cipher algorithm may be a symmetric or an asymmetric cipher.

In step 805, the Zero-Power, Energy-Harvesting Computational Module 250 also computes the response (R′), using the same function, and compares the computed response (R′) to the response received from the external device or Interrogator 630 (R). If the two response values match, as in step 806, the authentication is successful. In a preferred embodiment, the Zero-Power, Energy-Harvesting Computational Module 250 returns a “not authenticated” status to Interrogator 630 if the two response values do not match and an “authenticated” status to Interrogator 630 if the two response values match. Once authenticated, communications between the Interrogator 630 and the Zero-Power, Energy-Harvesting Computational Module 250 may proceed as described above and shown in FIGS. 7A and 7B.

The claims should not be read as limited to the described order or elements unless stated to that effect. Therefore, all embodiments that come within the scope and spirit of the following claims and equivalents thereto are claimed as the invention.

What is claimed:

1. A system comprising:
a device comprising at least one integrated circuit, wherein the device is vulnerable to unauthenticated access;
an antenna, and;
a zero-power, energy-harvesting reprogrammable computational module configured to communicate with the antenna to receive radio frequency signals and to communicate with the device,
wherein the computational module is powered by a corresponding radio frequency signal and authenticates the source of the corresponding radio frequency signal using a secure challenge-response cryptographic function.

2. The system of claim 1 further comprising an interrogator configured to transmit the corresponding radio frequency signal.

3. The system of claim 1, where the device further comprises a battery.

4. The system of claim 1, where the antenna is integrated with the computational module.

5. The system of claim 1, where the device comprises non-volatile memory.

6. The system of claim 1, where the device comprises a microcontroller having a data bus and non-volatile memory.

7. The system of claim 1 further comprising:
a UHF transmitter or transceiver in communication with the device; and
a UHF antenna in communication with the UHF transmitter or transceiver.

8. The system of claim 1, where the computational module comprises a microcontroller.

9. The system of claim 1, where the computational module is hard-wired to the vulnerable device.

10. The system of claim 1, where the computational module communicates wirelessly with the device.

11. The system of claim 2, where the computational module and the interrogator communicate using a RFID protocol.

12. The system of claim 11, where the RFID protocol is an Electronic Product Code (EPC) Class 1 Generation 1 protocol, EPC Class 1 Generation 2 protocol, ISO/IEC 7816, ISO/IEC 14443 or ISO/IEC 18092.

13. The system of claim 1, where the radio frequency signal is an ultra high frequency (UHF) signal.

14. The system of claim 1, where the radio frequency signal is a high frequency (HF) signal.

15. The system of claim 1, where the radio frequency signal is a low frequency (LF) signal.

16. The system of claim 1, where the radio frequency signal is a medical implant communications service (MICS) signal.

17. The system of claim 1, where the device is an implantable medical device.

18. The system of claim 1, where the system is incorporated in an automobile key.

19. The system of claim 1, where the system is incorporated in a key fob.

20. The system of claim 1, where the system is incorporated in a building access card or a room access card.

21. The system of claim 1, where the system is incorporated in a contactless smart card.

22. The system of claim 1, where the system is incorporated in a travel document, a driver's license, a personal identity verification card, a medical identity card or an employee identity card.

23. The system of claim 1, where the source of the corresponding radio frequency signal is authenticated using a cryptographic function.

24. The system of claim 23, where the cryptographic function is a symmetric cryptographic function.

25. The system of claim 23, where the cryptographic function is an asymmetric cryptographic function.

26. The system of claim 1, further comprising the step of sending a unique identifier to the source of the corresponding radio frequency signal.

27. The system of claim 26, further comprising the step of sending a nonce to the source of the corresponding radio frequency signal.

28. The system of claim 27, further comprising the step of computing a key value using the unique identifier and the nonce.

29. The system of claim 26, further comprising the step of computing a key value using the unique identifier.

30. The system of claim 29, further comprising the step of comparing the computed key value to a key value received from the source of the corresponding radio frequency signal.

31. The system of claim 29, where the key value is computed using a cryptographic function.

32. The system of claim 29, where the key value is computed using an Advanced Encryption Standard (AES) algorithm.

33. The system of claim 1, further comprising sending an authentication status to the source of the corresponding radio frequency signal.

34. A method of secure radio frequency communication with a vulnerable device, comprising the steps of:
providing a system comprising a device comprising at least one integrated circuit, wherein the device is vulnerable to unauthenticated access; an antenna, and; a zero-power, energy-harvesting reprogrammable computational module configured to communicate with the antenna to receive radio frequency signals and to communicate with the device, wherein the computational module is powered by a corresponding radio frequency signal and authenticates the source of the corresponding radio frequency signal using a cryptographic function;
using the system to receive a corresponding radio frequency signal from an interrogator
storing the energy in the received corresponding radio frequency signal;
authenticating the interrogator using an encrypted challenge-response authentication mechanism between the zero-power, energy-harvesting reprogrammable computational module and the interrogator; and
enabling communication between the interrogator and the device if the interrogator is authenticated.

35. The method of claim 34, where the device further comprises a battery.

36. The method of claim 34, where the antenna is integrated with the computational module.

37. The method of claim 34, where the device comprises non-volatile memory.

38. The method of claim 34, where the device comprises a microcontroller having a data bus and non-volatile memory.

39. The method of claim 34 further comprising:
a UHF transmitter or transceiver in communication with the device; and
a UHF antenna in communication with the UHF transmitter or transceiver.

40. The method of claim 34, where the computational module comprises a microcontroller.

41. The method of claim 34, where the computational module is hard-wired to the enabled device.

42. The method of claim 34, where the computational module communicates wirelessly with the device.

43. The method of claim 34, where the computational module and the interrogator communicate using a RFID protocol.

44. The method of claim 32, where the RFID protocol is an Electronic Product Code (EPC) Class 1 Generation 1 protocol, EPC Class 1 Generation 2 protocol, ISO/IEC 7816, ISO/IEC 14443 or ISO/IEC 18092.

45. The method of claim 34, where the radio frequency signal is an ultra high frequency (UHF) signal.

46. The method of claim 34, where the radio frequency signal is a high frequency (HF) signal.

47. The method of claim 34, where the radio frequency signal is a low frequency (LF) signal.

48. The method of claim 34, where the radio frequency signal is a medical implant communications service (MICS) signal.

49. The method of claim 34, where the device is an implantable medical device.

50. The method of claim 34, where the system is incorporated in an automobile key.

51. The method of claim 34, where the system is incorporated in a key fob.

52. The method of claim 34, where the system is incorporated in a building access card or a room access card.

53. The method of claim 34, where the system is incorporated in a contactless smart card.

54. The method of claim 34, where the system is incorporated in a travel document, a driver's license, a personal identity verification card, a medical identity card or an employee identity card.

55. A method for communicating with an implantable medical device (IMD) comprising the steps of:
receiving a radio frequency (RF) signal from an interrogator, where the RF signal comprises a command for the IMD;
harvesting energy from the RF signal; and
sending the command to the IMD.

56. The method of claim 55, further comprising the step of determining if there is sufficient harvested energy before sending the command to the IMD.

57. The method of claim 55, further comprising storing energy harvested from the RF signal.

58. The method of claim 55, further comprising receiving a reply from the IMD.

59. The method of claim 55, further comprising sending a response to the interrogator.

60. The method of claim 57, further comprising performing a computation before sending the response to the interrogator.

61. A method for securely communicating with an implantable medical device (IMD) comprising the steps of:
receiving an RF signal comprising an authentication request from an interrogator;
authenticating the interrogator using energy harvested from the RF signal; and
permitting access to the IMD if the interrogator is authenticated.

62. The method of claim 61, where the interrogator is authenticated using a cryptographic function.

63. The method of claim 51, where the cryptographic function is a symmetric cryptographic function.

64. The method of claim 51, where the cryptographic function is an asymmetric cryptographic function.

65. The method of claim 61, further comprising the step of sending a unique identifier to the interrogator.

66. The method of claim 65, further comprising the step of sending a nonce to the interrogator.

67. The method of claim 66, further comprising the step of computing a key value using the unique identifier and the nonce.

68. The method of claim 65, further comprising the step of computing a key value using the unique identifier.

69. The method of claim 68, further comprising the step of comparing the computed key value to a key value received from the interrogator.

70. The method of claim 68, where the key value is computed using a cryptographic function.

71. The method of claim 68, where the key value is computed using an Advanced Encryption Standard (AES) algorithm.

72. The method of claim 61, further comprising sending an authentication status to the interrogator.

73. The method of claim 61, further comprising receiving a command for the IMD.

74. A system comprising:
an electronic lock comprising an interrogator;
a zero-power, energy-harvesting computational module configured to communicate with the electronic lock; and
where the interrogator wirelessly transmits a radio frequency (RF) signal to the computational module, where the computational module is powered by the incoming RF signal.
